(19) United States
(12) Patent Application Publication
(10) Pub. No.: US 2011/0123600 A1
(43) Pub. Date: May 26, 2011

(54) Use of alkylureas for treating acne

(76) Inventor: Arye Rubinstein, Wesley Hills, NY (US)

(21) Appl. No.: 12/927,102

(22) Filed: Nov. 5, 2010

Related U.S. Application Data
(63) Continuation of application No. 11/049,377, filed on Feb. 2, 2005, now abandoned.

(51) Int. Cl. A61K 31/17; C07C 275/06; A61K 9/127; A61P 17/10

(52) U.S. Cl. 424/450; 514/588; 564/58

(57) ABSTRACT

Methods of treating acne are provided. The methods involve applying to the acne an alkyl urea in a pharmaceutically acceptable carrier. The use of an alkyl urea in the manufacture of a medicament for the treatment of acne is also provided. Compositions of alkyl ureas in a pharmaceutically acceptable carrier are additionally provided.

### CROSS REFERENCE TO RELATED APPLICATIONS

This application is a continuation of U.S. application Ser. No. 11/049,377, filed Feb. 2, 2005, which claims the benefit of PCT Application No. PCT/US2003/023916, filed Jul. 31, 2003, which claims the benefit of U.S. Provisional Application No. 60/400,518, filed Aug. 2, 2002, the contents of which are hereby incorporated by reference in their entirety.

### BACKGROUND OF THE INVENTION

1. Field of the Invention

The present invention generally relates to a treatment for skin disorders involving the pilosebaceous units of the skin. The invention also relates to a treatment of skin disorders such as acne vulgaris, acne conglobara, acne fulminans, and comedonal acne.

Acne is an inflammation of the pilosebaceous units of certain body areas such as the face, neck, trunk, and buttocks. Acne often manifests itself as comedones (comedonal acne), papulopustular acne), or nodules and cysts (nodulocystic acne and acne conglobara). Pitted, depressed, or hypertrophic scars may follow all types of acne, but especially nodulocystic acne and acne conglobara. Severe acne often includes one or more of the following: persistent or recurrent inflammatory nodules, extensive papulopustular lesions, active scarring, and/or presence of sinus tracts.

Acne disorders are often classified as noninflammatory or inflammatory types. Noninflammatory acne is characterized by closed comedones (whiteheads) and open comedones (blackheads), consisting of compact masses of keratin, sebum, and bacteria which dilate the follicular duct. Inflammatory acne is characterized by papules (pimples), pustules, and nodulocystic lesions which may lead to scarring.

Several factors are believed to play important roles in the pathogenesis of acne including: hormonal stimulation, plugged pores and skin pathogens (i.e., Propioni bacterium acnes).

### A. Hormonal Stimulation

Androgens cause the sebaceous glands to mature and produce large quantities of sebum, which explains, in part, why acne is seen most during puberty. In the male, the primary source of androgens is the testes, whereas in the female, the ovaries and adrenals produce these hormones.

Since sebaceous glands are extremely sensitive to androgenic stimulation, it is not surprising that females, even with relatively low levels of circulating androgens, can have as much trouble with acne as males. Also certain androgen secreting tumors of the adrenals and ovaries may cause acne vulgaris, often in association with signs of masculinization in the female, or prepubertal child. Women with the Stein-Leventhal syndrome (polycystic ovaries) have an increased production of testosterone by the ovaries and often have a severe case of acne as well. Acne may also be seen in patients receiving exogenous androgens, as in body builders taking steroids.

### B. Plugged Pores

Blackheads and whiteheads, more properly called comedones, are the hallmark of acne vulgaris and are thought to be the initial lesion in acne. They represent inspissated sebum and keratin debris filling and blocking the sebaceous gland opening. By obstructing the flow of sebum, normal function is altered and the gland is rendered more susceptible to inflammation. This results from the physical stretching of the follicle walls by the enlarging comedone, and the stagnating sebum which is unable to exit. This sebum serves as an inviting culture media for resident bacteria. With resulting leakage of irritating components into the surrounding dermis through these compromised follicular walls, an inflammatory response begins to be mounted. After this first insult, the other familiar clinical features of a full blown acne lesion follow, namely the papule, pustule, and cyst.

### C. Skin Pathogens

The third factor contributing to the pathogenesis of acne is the gram positive bacillus, Propioni bacterium acnes, which is found in the follicles and sebaceous ducts of patients both with and without acne. Sebum synthesized by the sebaceous gland is almost pure triglyceride and is non-irritating. If triglyceride was the only lipid leaking into the dermis due to comedo obstruction, little inflammation would occur. However, Propioni bacterium acnes is a bacterium that metabolizes triglycerides. More specifically, it transforms the triglycerides into irritating essential fatty acids via a lipase enzyme system. These free fatty acids make up the principal irritants initiating the inflammatory process in acne.

Several other factors go into the pathogenesis of acne. Genetic susceptibility, environmental influences, certain medications, cosmetics, stress, fatigue and of course, picking at one's lesions may all play a role or aggravate existing acne. Efforts to limit these contributors can help control the active disease.

While superficial acne is responsive to current topical therapies, deep acne requires vigorous management which may include oral antibiotics and Isotretinoin (acutane). Minocycline is the most effective antibiotic. Antibiotics are additive and should not be the sole treatment or the beginning treatment.

Tetracycline may have an effect on gram negative bacteria that colonize the sebaceous glands (e.g. Propioni Bacterium acnes). Long term use does, however, produce a gram negative folliculitis which is difficult to clear. In addition the use of antibiotics reduces the number of new inflamed cysts but has little effect on existing cysts.

Topical retinoids (tretinoin, Retin-A^{R}, Avita^{R}) are effective in comedonal and papulopustular acne but require detailed instructions regarding usage and potential side effects including:
1. Teratogenic effects.
2. Skin irritation including edema, redness, blistering, crust formation, peeling, pain.
3. Temporary skin hyper or hypo pigmentation.
4. Heightened sensitivity to sunlight.

All of the potential adverse side effects of topical retinoids can occur with oral Isotretinoin. Because it is teratogenic, pregnancy should be prevented. Both tetracycline and Isotretinoin may cause pseudo-tumor cerebri (benign intra-cranial swelling); therefore, the two medications should never be used together. About 25% of patients can develop increased triglycerides. Also, 15% of patients develop a decrease in high-density lipoproteins (HDL), and about 7% show an increase in cholesterol levels. This may increase cardiovascular risk. Also when levels of serum triglycerides rise above 800 mg/ul, the patient may develop acute pancreatitis. Elevation may occur in Hepatic enzymes. Also night blindness has been reported as well as decreased tolerance to contact lenses.

There is, therefore, a need for a topical product that can address these problems effectively without minimal, if any, adverse effects.

2. Description of Related Art

The following references provide further background to the present invention. The references also point out many shortcomings which the present invention overcomes. The references are incorporate herein by references in their entirety.
1) Eichenfeld et al. Acne: Current concepts of pathogenesis and approach to rational treatment. Pediatrician 18:218, 1991.
Winston M H, Shalita A R, Acne vulgaris. Pediatr. Clin. North Am. 38:889, 1991.
3) Elbaum D L, Herskovits T T. Dissociation of human hemoglobin by the ureas and amides. Osmotic pressure and light scattering studies. Biochemistry, 13:1268-1278, 1974.
4) Tanford C. Protein denaturation. Adv. Prot. Chem. 23:121-282, 1968.
5) Warren J R, and Gordon J A. The nature of alkylurea and urea denaturation of a chymotrypsinogen. Biochem. Biophys. Acta 420:397-405, 1976.
6) Elbaum D, Pandolfelli E R, and Herskovits T T. Denaturation of human and Glyceria dibranchiata hemoglobulins by the urea and amide class of denaturants. Biochemistry 13:1278-1284, 1974.
7) Goldstein A, Pettoello-Manbtovani M, Kollmann T R, Calvelli T A, Rubinstein A. Inhibition of HIV-1 infection by alkylureas. AIDS 5:1447-1452, 1991.
8) Dar M S, and Fakouhi T. Studies on some aspects of the neuropharmacology of butylurea. Arch. Intern. De Pharmacodynamie et de Therapie 208(2):204-212, 1974.

Many features, advantages and objects of the present invention will become apparent to one with skill in the art upon examination of the detailed description. It is intended that all such features, advantages, and objects be included herein within the scope of the present invention.

### DETAILED DESCRIPTION OF THE PRESENT INVENTION

In preliminary studies, alkyl urea containing compounds were able to rapidly cure superficial as well as deep acne. An alkyl urea is a non-aromatic amide. The basic formula for an alkyl urea is R—HN—CO—NH₂. “R” represents an alkyl group. The term “alkyl” refers to a monovalent alkane (hydrocarbon) derived radical group.

The alkyl group of the present invention includes both short chain and medium chain alkyl groups. The term “short chain alkyl” or “lower alkyl” refers to an alkyl group of 1 to 6 carbon atoms. “Medium chain alkyl” groups refer to an alkyl group of 7 to 12 carbon atoms. The alkyl groups of the present invention may be a straight chain, branched, or cycloalkyl groups. Specifically, the alkyl groups of the present invention contain from 1 to 8 carbon atoms. Examples of alkyl groups which may be used with the present invention include, but are not limited to the following: methyl, ethyl, poropyl, isopropyl, n-butyl, I-butly, isobutyl, pentyl, hexyl, isohexyl, heptyl, and octyl.

The alkyl component of an alkyl urea is non-polar and hydrophobic. It should be noted that hydrophobic forces are an important determinant of protein structure, folding and stability. The urea component, (—NH—CO—NH₂), is polar and hydrophilic.

The combination of non-polar and polar moieties is referred to as amphiphilic or amphipathic in character. These ureas have surfactant properties, and are considered to be Class II Additives having hydrogen bonding properties.

All stereoisomers of the compounds of the present invention are contemplated and within the scope of the invention, either in admixture or in pure or substantially pure form. The definition of the compounds according to the invention embraces all possible stereoisomers and their mixtures. It very particularly embraces the racemic forms and the isolated optical isomers having the specified activity. The racemic forms can be resolved by physical methods such as, for example, fractional crystallization, separation or crystallization of diastereomeric derivatives or separation by column chromatography. The individual optical isomers can be obtained from the racemates by conventional methods, such as, for example, salt formation with an optically active acid followed by crystallization.

One example of an alkyl urea of the present invention is butyl urea (BU). It is a mono-substituted lower alkyl urea. The chemical formula for butyl urea is:

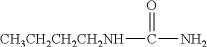

BU has a molecular weight of 116.6 and a melting point of 96-98° C. Short chain and medium chain alkyl urea compounds uniquely combine the keratolytic/hydrating properties of ureas, with the lipid disrupting abilities of the non-polar alkyl groups. The effect of BU on acne may be related to a variety of interrelated factors:
1. BU is a small bipolar molecule with a hydrophobic and hydrophilic “pole.” By this token it intercalates into lipophilic cell walls and may dissolve pilosebaceous secretions.
2. Through its hydrophilic characteristics BU is highly hygroscopic and rapidly increases fluid retention in the sebaceous cyst accelerating the secretion of fluid from the acne lesion.
3. BU is in the used concentration toxic to lymphocytes, neutrophils and macrophages and thus decreasing the local inflammatory reaction in the acne cyst.
4. BU has direct anti viral and bacterial properties. A 5-6 log reduction in bacterial counts was noted with 0.4 M BU on E. Coli, Pseudomonas aeroginosa, Proteus mirabilis and Proteus vulgaris. The mechanisms by which BU might kill bacteria may also be responsible for its other favorable effects on acne. Urea and alkylureas such as BU can denature proteins by altering their tertiary structure. Urea predominantly denatures proteins by affecting interactions mediated by hydrogen bond formation of peptide groups while alkylureas alter protein conformational structures by disrupting hydrophobic forces. The concentration of an alkylurea required to denature most soluble proteins is significantly less than that of urea and depends on the length of the alkyl chain. In addition, alkylureas such as BU denature membrane proteins more potently than they denature soluble proteins because the increased lipid solubility conferred by the hydrophobic alkyl chain allows them to intercalate into the membrane. Finally, molecules which prefer the hydrophobic regions of lipid bilayer membranes and hydrophobic domains of proteins will also prefer the hydrophobic regions of nucleic acids. Preferential binding to nucleic acids may explain the sparing by BU of red blood cells that do not contain nucleic acids, whereas viruses and bacteria that do not have nucleic acids are affected.

The alkyl ureas of the present invention may be used alone or in various concentrations with any pharmaceutically acceptable carrier. As an example, the alkyl ureas may be topically applied in a non-toxic-hypoallergenic pharmaceutical carrier, such as aqueous solution of distilled water, or as a solution in a non-toxic polar organic solvent or as a suspension or as an emulsion in a non-toxic cream composition or non-toxic polymer gel. As an example, a preferred range of concentration of BU in an aqueous solution is from 0.1 molar (1.2% w/v) to 0.5 molar (5.8% w/v).

Topical alkyl ureas may be incorporated into a vehicle containing liposomes. Liposomes are comprised of phospholipd groups. Liposomal vesicles may be formed when combining liposomes with an aqueous solution. Liposomes may vary in shape and size and may be designed with specific characteristics. Liposomes can be used to target specific areas, control the pharmacokinetics of a medication, or to enhance efficacy of a medication. Liposomal bases which may be used in the present invention are available from a variety of vendors.

Examples of suitable non-toxic polar or organic solvents include lower alkanols of 2 to 6 carbon atoms, such as ethanol, lower alkanetriols of 3 to 6 carbon atoms, such as glycerol, and alkanehexols, such as sorbital.

Examples of suitable non-toxic cream compositions include a suspension or an emulsion of the alkyl urea in a non-toxic, non-hydrophobic cream, for example, a silicone emulsion or a polyethylene-glycol emulsion.

Topically, the composition may be applied in various types of applications. I.e. rollerball, perforated dome tipped tube or cylinder, or transdermal patch.

The present invention is illustrated by the following examples that should not be considered limiting.

### Example 1

BU in polymer gels: BU incorporated in a polymer gel designed to release the chemical within 30 minutes to 3 hours was administered topically to 11 patients with inflamed cystic lesions of acne. The BU end concentration to be released was between 0.4 to 0.5 molar. Applications to the lesions alone were made 3× daily for 1 week. In all 11 patients no local adverse reactions were noted. Most of the lesions resolved by the end of the treatment. The purulent discharge as well as the inflammatory component of all lesions resolved often within 24 hours.

### Example 2

BU in an squeous solution: A 0.5 molar (5.8% w/v) solution of BU was applied twice daily with Q tips to acne lesions in 5 adolescents. BU was applied to cystic purulent lesions, to non cystic lesions with erythema and to early lesions with minimal inflammation. The inflammatory response resolved in all lesions within 24-48 hours, whereas purulent cystic lesions healed without scarring within 3-7 days. These observations suggest that the anti-inflammatory effects of BU may be the main or at least the earliest mechanism responsible for BU's therapeutic effect and precede the antibacterial effects.

### Example 3

Preparation of Sample: 11.6 grams of butyl urea (BU) sample was weighed accurately and dissolved in de-ionized water to 100 ml into 100 ml volumetric flask and mixed to form a 1M (11.6% w/v) solution. Then, 10.0 ml of the 1M solution was mixed with 12.7 ml de-ionized water to form a 0.44M (5.1% w/v) solution. (10.0 ml×1.0M÷22.7 ml=0.44M). Finally, the 0.44M solution was filtered through sterile syringe filters to sterilize.

Procedure: For the 0.4M butylurea solutions, 4.5 ml was aliquoted into seven (7) 16×150 mm sterile glass test tubes. 4.5 ml sterile physiological saline was also aliquoted into seven (7) 16×150 ml sterile glass test tubes as a control.

0.6 ml of culture suspension was inoculated into 0.44M BU and saline control tube for each organism tested and mixed. After one hour contact time a 1.0 ml sample culture mixer was diluted serially into 10⁶ into 9.0 ml GBL Stat Broth (TSB; 0.5% Leethen; 4% Tween 20 and 0.1% Na₃S₂O₃).

One ml Aerobic Plate Count was performed from each dilution tubes using Trypticase Soy Agar. Plates were incubated at 37±° C. for 48 hours. Except Candida albicans plates were poured with Sabourad Dextrose Agar and incubated at 20 to 25° C. for 5 to 7 days. Colonies were counted using a Quebec Colony Counter.

Final Concentration of Butylurea:
4.5 ml × 0.44M + 5 ml = 0.4M

| Organism | 0.4M BU |
|---|---|
| Staphylococcus aureus | Not Active |
| Streptococcus pyogenes | 2 log Reduction |
| Escherichla coli | 6 log Reduction |
| Candida albicans | 0.4 log Reduction |
| Pseudomonas aeruginosa | 5 log Reduction |
| Proteus mirablis | 5 log Reduction |
| Proteus vulgaris | 6 log Reduction |

It should be emphasized that the foregoing description has been presented for purpose of providing a clear understanding of the invention. The description is not intended to be exhaustive or to limit the invention to the precise examples disclosed. Obvious modifications or variations by one with skill in the art are possible in light of the above teachings without departing from the spirit and principles of the invention. All such modifications and variations are intended to be within the scope of the present invention.

What is claimed is:

1. A method of treating acne on the skin of a patient, the method comprising applying to the acne an alkyl urea in a pharmaceutically acceptable carrier at a concentration and for a time sufficient to reduce the acne,
wherein the alkyl urea has the formula R—HN—CO—NH₂, where R is a monovalent alkane of 1-12 carbon atoms.

2. The method of claim 1, wherein R has 1-8 carbon atoms.

3. The method of claim 1, wherein the alkyl urea is butyl urea.

4. The method of claim 1, wherein the alkyl urea is at a concentration of 0.1 molar to 0.5 molar.

5. Use of an alkyl urea for the manufacture of a medicament for the treatment of acne, wherein the alkyl urea has the formula R—HN—CO—NH₂, where R is a monovalent alkane of 1-12 carbon atoms.

6. The use of claim 5, wherein R has 1-8 carbon atoms.

7. The use of claim 5, wherein the alkyl urea is butyl urea.

8. The use of claim 5, wherein the alkyl urea is at a concentration of 0.1 molar to 0.5 molar.

9. An alkyl urea in a pharmaceutically acceptable carrier, at a concentration useful for the treatment of acne, wherein the alkyl urea has the formula R—HN—CO—NH₂, where R is a monovalent alkane of 1-12 carbon atoms.

10. The alkyl urea of claim 9, wherein R has 1-8 carbon atoms.

11. The alkyl urea of claim 9, wherein the alkyl urea is butyl urea.

12. The alkyl urea of claim 9, wherein the alkyl urea is at a concentration of 0.1 molar to 0.5 molar.

13. The alkyl urea of claim 9, wherein the carrier is selected from the group consisting of distilled water, a polar organic solvent, a cream, a gel, and a liposome.
